(19) United States
(12) Patent Application Publication
(10) Pub. No.: US 2024/0390028 A1
(43) Pub. Date: Nov. 28, 2024

(54) CUTTING BLADE WITH SHORTENED TERMINAL BLADE SEGMENTS

(71) Applicant: Boston Scientific Scimed, Inc., Maple Grove, MN (US)

(72) Inventor: Gordon John Kocur, Lino Lakes, MN (US)

(73) Assignee: Boston Scientific Scimed, Inc., Maple Grove, MN (US)

(21) Appl. No.: 18/670,955

(22) Filed: May 22, 2024

Related U.S. Application Data
(60) Provisional application No. 63/468,615, filed on May 24, 2023.

(51) Int. Cl. A61B 17/3207; A61B 17/22; A61M 25/10

(52) U.S. Cl. CPC A61B 17/320725; A61B 2017/22061; A61M 25/1018

(57) ABSTRACT

A medical device useful for cracking or cutting an intravascular lesion may include a monolithic blade or a blade assembly including a first terminal blade segment and a second terminal blade segment. The length of each of the first terminal blade segment and the second terminal blade segment, which may be defined at least in part by one or more flex points, may allow the first terminal blade segment and the second terminal blade segment to flex at least 30 degrees relative to a longitudinal axis when the monolithic blade is pressed against a lesion.

### CROSS-REFERENCE TO RELATED APPLICATIONS

This application claims the benefit of U.S. Provisional Patent Application Ser. No. 63/468,615, filed May 24, 2023, which is incorporated herein by reference.

### TECHNICAL FIELD

The disclosure is directed to angioplasty balloon catheters including one or more cutting blades mounted to the balloon. More particularly, the disclosure is directed to cutting blades of a cutting balloon catheter having shortened terminal blade segments.

### BACKGROUND

Heart and vascular disease are major problems in the United States and throughout the world. Conditions such as atherosclerosis result in blood vessels becoming blocked or narrowed. This blockage can result in lack of oxygenation of the heart, which has significant consequences since the heart muscle must be well oxygenated in order to maintain its blood pumping action, or lack of oxygenation and/or circulation to other regions of the body.

Occluded, stenotic, or narrowed blood vessels, as well as native or synthetic arteriovenous dialysis fistulae, may be treated in a recanalization procedure, such as with an angioplasty balloon catheter advanced over a guidewire to an occlusion so that the balloon is positioned across the occlusion. The balloon is then inflated to enlarge the passageway through the occlusion.

One of the major obstacles in treating coronary artery disease and/or treating blocked blood vessels or fistulae is re-stenosis or re-narrowing of the passageway through the occlusion subsequent to an angioplasty procedure or other recanalization procedure. Evidence has shown that cracking, cutting or scoring the stenosis, for example, with an angioplasty balloon equipped with a blade member, during treatment can reduce incidence of re-stenosis. Additionally, cracking, cutting or scoring the stenosis may reduce trauma at the treatment site and/or may reduce the trauma to adjacent healthy tissue. Blade members may also be beneficial additions to angioplasty procedures when the targeted occlusion is hardened or calcified. It is believed typical angioplasty balloons, alone, may not be able to expand certain of these hardened lesions. Thus, angioplasty balloons equipped with blade members have been developed to attempt to enhance angioplasty treatments. Blade members may be adapted to cut, crack or score lesions, for example. While these blade members may include flex points that allow the blade member to bend, there is an ongoing need for blade members that are adapted to permit further flexing of the blade member, so that the terminal end segments of the blade member can flex to a greater angle relative to an end of a calcified portion of a lesion, as well as to permit delivery through tortuous portions of the vasculature.

### SUMMARY

The disclosure is directed to several alternative designs, materials and methods of manufacturing medical device structures and assemblies, and the use thereof. An example may be found in a medical device that includes a catheter shaft having a distal region and an inflatable balloon that is secured to the distal region. A monolithic blade member extends longitudinally relative to the inflatable balloon and is the monolithic blade member secured relative to an outer surface of the inflatable balloon. The monolithic blade member includes a base portion extending from a first end of the monolithic blade member to a second end of the monolithic blade member and a blade portion extending outwardly from the base portion. The blade portion includes a first terminal blade segment extending from the first end of the monolithic blade member to a first flex point formed in the monolithic blade member, the first terminal blade segment having a first terminal blade segment length, a second terminal blade segment extending from the second end of the monolithic blade member to a second flex point formed in the monolithic blade member, the second terminal blade member having a second terminal blade segment length, and at least one intermediate blade segment extending between the first flex point and the second flex point, each of the at least one intermediate blade segments having an intermediate blade segment length that is greater than the first terminal blade member segment length and the second terminal blade member segment length.

Alternatively or additionally, the terminal blade segment length may allow the first flex point to be within two millimeters of a first end of a lesion when the second flex point is within two millimeters of an opposing second end of the lesion.

Alternatively or additionally, the first flex point may include a first gap in the blade portion and the second flex point may include a second gap in the blade portion, with the base portion extending without any gap.

Alternatively or additionally, the first terminal blade segment length may be equal to the second blade segment length with the base portion extending without any gap.

Alternatively or additionally, the first terminal blade segment may have a length of less than 3 millimeters and the second terminal blade segment may have a length of less than 3 millimeters.

Alternatively or additionally, the first terminal blade segment may have a length of less than 2 millimeters and the second terminal blade segment may have a length of less than 2 millimeters.

Alternatively or additionally, the medical device may further include one or more additional monolithic blade members extending longitudinally along the outer surface of the inflatable balloon. Each of the one or more additional monolithic blade members may include a base portion extending from a first end of the monolithic blade member to a second end of the monolithic blade member and a blade portion extending outwardly from the base portion. The blade portion may include a first terminal blade segment extending from the first end of the monolithic blade member to a first flex point formed in the monolithic blade member, the first terminal blade segment having a first terminal blade segment length, a second terminal blade segment extending from the second end of the monolithic blade to and a second flex point formed in the monolithic blade member, the second terminal blade segment having a second terminal blade segment length, and at least one intermediate blade segment extending between the first flex point and the second flex point, the at least one intermediate blade segment having an intermediate blade segment length that is greater than the first terminal blade segment length and the second terminal blade segment length.

Another example may be found in a medical device that includes an inflatable balloon secured to a distal region of a catheter shaft having a longitudinal axis and a plurality of blade assemblies secured to the inflatable balloon and extending longitudinally with respect to the inflatable balloon. Each of the plurality of blade assemblies have a longitudinal axis and include a polymeric pad adapted to be secured to the inflatable balloon and a plurality of blade segments secured within the polymeric pad. The plurality of blade segments includes a first terminal blade segment having a length of less than 3 millimeters at a first end of the blade assembly, a second terminal blade segment having a length of less than 3 millimeters at an opposing second end of the blade assembly, and at least one intermediate blade segment disposed between the first terminal blade segment and the second terminal blade segment.

Alternatively or additionally, the polymeric pad may include a single polymeric pad, with each of the plurality of blade segments aligned axially with respect to the single polymeric pad and secured within the single polymeric pad.

Alternatively or additionally, the polymeric pad may include a plurality of individual polymeric pad segments, with each blade segment of the plurality of blade segments secured within an individual polymeric pad segment of the plurality of individual pad segments.

Alternatively or additionally, the first terminal blade segment may be positioned relative to an adjacent intermediate blade segment such that first terminal blade segment is able to be flexed at an angle greater than 30 degrees from the longitudinal axis and the second terminal blade segment may be positioned relative to an adjacent intermediate blade segment such that the second terminal blade segment is able to be flexed at an angle greater than 30 degrees from the longitudinal axis.

Alternatively or additionally, the first terminal blade segment may be positioned relative to an adjacent intermediate blade segment such that the first terminal blade segment is able to be flexed at an angle greater than 45 degrees from the longitudinal axis and the second terminal blade segment may be positioned relative to an adjacent intermediate blade segment such that the second terminal blade segment is able to be flexed at an angle greater than 45 degrees from the longitudinal axis.

Alternatively or additionally, the at least one intermediate blade segment may extend parallel to the longitudinal axis while the first terminal blade segment and the second terminal blade segment are flexed at an angle greater than 30 degrees from the longitudinal axis.

Another example may be found in a method for treating a lesion within a blood vessel. The method includes advancing a medical device through the blood vessel to a position proximate the lesion, the medical device including an inflatable balloon and a monolithic blade including a base portion extending from a first end of the monolithic blade member to a second end of the monolithic blade member and a blade portion extending outwardly from the base portion. The blade portion includes a first blade segment extending from the first end of the monolithic blade member to a first flex point formed in the monolithic blade member, a second blade segment extending from the second end of the monolithic blade member to a second flex point formed in the monolithic blade member, and a third blade segment extending between the first blade segment and the second blade segment. The method includes positioning the medical device such that the first blade segment extends distally of the lesion and the second blade segment extends proximally of the lesion. The inflatable balloon is inflated in order to exert the monolithic blade towards the lesion, at least one of the first blade segment and the second blade segment flexing to an angle greater than 30 degrees relative to the third blade segment.

Alternatively or additionally, at least one of the first blade segment and the second blade segment may flex to an angle greater than 45 degrees with respect to the third blade segment when the inflatable balloon is inflated.

Another example may be found in a medical device including a catheter shaft having a distal region and an inflatable balloon secured to the distal region. A monolithic blade member extends longitudinally relative to the inflatable balloon and is secured relative to an outer surface of the inflatable balloon. The monolithic blade member includes a base portion extending from a first end of the monolithic blade member to a second end of the monolithic blade member and a blade portion extending outwardly from the base portion. The blade portion includes a first terminal blade segment extending from the first end of the monolithic blade member to a first flex point formed in the monolithic blade member, the first terminal blade segment having a first terminal blade segment length, a second terminal blade segment extending from the second end of the monolithic blade member to a second flex point formed in the monolithic blade member, the second terminal blade member having a second terminal blade segment length, and at least one intermediate blade segment extending between the first flex point and the second flex point, each of the at least one intermediate blade segments having an intermediate blade segment length that is greater than the first terminal blade member segment length and the second terminal blade member segment length.

Alternatively or additionally, the terminal blade segment length may allow the first flex point to be within two millimeters of a first end of a lesion when the second flex point is within two millimeters of an opposing second end of the lesion.

Alternatively or additionally, the first flex point may include a first gap in the blade portion.

Alternatively or additionally, the second flex point may include a second gap in the blade portion, with the base portion extending without any gap.

Alternatively or additionally, the first terminal blade segment length may be equal to the second blade segment length with the base portion extending without any gap.

Alternatively or additionally, the first terminal blade segment may have a length of less than 3 millimeters.

Alternatively or additionally, the first terminal blade segment may have a length of less than 2 millimeters.

Alternatively or additionally, the second terminal blade segment may have a length of less than 2 millimeters.

Alternatively or additionally, the second terminal blade segment may have a length of less than 3 millimeters.

Alternatively or additionally, the medical device may further include one or more additional monolithic blade members extending longitudinally along the outer surface of the inflatable balloon. Each of the one or more additional monolithic blade members include a base portion extending from a first end of the monolithic blade member to a second end of the monolithic blade member and a blade portion extending outwardly from the base portion. The blade portion includes a first terminal blade segment extending from the first end of the monolithic blade member to a first flex point formed in the monolithic blade member, the first terminal blade segment having a first terminal blade segment length, a second terminal blade segment extending from the second end of the monolithic blade to and a second flex point formed in the monolithic blade member, the second terminal blade segment having a second terminal blade segment length, and at least one intermediate blade segment extending between the first flex point and the second flex point, the at least one intermediate blade segment having an intermediate blade segment length that is greater than the first terminal blade segment length and the second terminal blade segment length.

Another example may be found in a medical device that includes an inflatable balloon secured to a distal region of a catheter shaft having a longitudinal axis. A plurality of monolithic blade members are secured to the inflatable balloon and extending longitudinally with respect the inflatable balloon. Each of the plurality of monolithic blade members include a base portion extending from a first end of the monolithic blade member to a second end of the monolithic blade member and a blade portion extending outwardly from the base portion. The blade portion includes a first terminal blade segment extending from the first end of the monolithic blade member to a first flex point formed in the monolithic blade member, the first terminal blade segment having a length of less than 3 millimeters, a second terminal blade segment extending from the second end of the monolithic blade member to a second flex point formed in the monolithic blade member, the second terminal blade segment having a length of less than 3 millimeters, and at least one intermediate blade segment extending between the first flex point and the second flex point, each of the at least one intermediate blade segments having a length of greater than 3 millimeters.

Alternatively or additionally, the plurality of monolithic blade members may include four blade members, each arranged circumferentially spaced about 90 degrees from a closest neighbor monolithic blade member.

Alternatively or additionally, for each of the plurality of monolithic blade members, the first pivot point may include a first gap in the blade portion.

Alternatively or additionally, for each of the plurality of monolithic blade members, the second pivot point may include a second gap in the blade portion, with the base portion extending without any gap.

Alternatively or additionally, the first flex point may be adapted to enable the first terminal blade segment to be flexed at an angle greater than 30 degrees from the longitudinal axis and the second flex point may be adapted to enable the second terminal blade segment to be flexed at an angle greater than 30 degrees from the longitudinal axis.

Alternatively or additionally, the first flex may be is adapted to enable the first terminal blade segment to be flexed at an angle greater than 45 degrees from the longitudinal axis.

Alternatively or additionally, the second flex point may be adapted to enable the second terminal blade segment to be flexed at an angle greater than 45 degrees from the longitudinal axis.

Alternatively or additionally, the at least one intermediate blade segment may extend parallel to the longitudinal axis while the first terminal blade segment and the second terminal blade segment are flexed at an angle greater than 30 degrees from the longitudinal axis.

Another example may be found in a method of treating a lesion within a blood vessel. The method includes advancing a medical device through the blood vessel to a position proximate the lesion, the medical device including an inflatable balloon and a monolithic blade including a base portion extending from a first end of the monolithic blade member to a second end of the monolithic blade member and a blade portion extending outwardly from the base portion. The blade portion includes a first blade segment extending from the first end of the monolithic blade member to a first flex point formed in the monolithic blade member, a second blade segment extending from the second end of the monolithic blade member to a second flex point formed in the monolithic blade member, and a third blade segment extending between the first blade segment and the second blade segment. The method includes positioning the medical device such that the first blade segment extends distally of the lesion and the second blade segment extends proximally of the lesion and inflating the inflatable balloon in order to exert the monolithic blade towards the lesion, at least one of the first blade segment and the second blade segment flexing to an angle greater than 30 degrees relative to the third blade segment.

Alternatively or additionally, at least one of the first blade segment and the second blade segment may flex to an angle greater than 45 degrees with respect to the third blade segment when the inflatable balloon is inflated.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a schematic partially cross-sectioned of an example medical device;

FIG. 2 is a schematic view of a monolithic blade member usable in the example medical device of FIG. 1;

FIG. 3 is a schematic view of a monolithic blade member usable in the example medical device of FIG. 1;

FIG. 4 is a schematic view of a monolithic blade member usable in the example medical device of FIG. 1;

FIG. 5 is a schematic view of a blade assembly usable in the example medical device of FIG. 1;

FIG. 6 is a schematic view of a blade assembly usable in the example medical device of FIG. 1;

FIG. 7 is a schematic view of a blade assembly usable in the example medical device of FIG. 1;

FIG. 8 is a schematic view showing the example medical device of FIG. 1 disposed within a blood vessel proximate a lesion, achieving a terminal blade segment flex of 30 degrees;

FIG. 9 is a schematic view showing the example medical device of FIG. 1 disposed within a blood vessel proximate a lesion, achieving a terminal blade segment flex of 45 degrees; and

FIG. 10 is a schematic view showing a relationship between blade member length and an achievable blood vessel radius of curvature.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

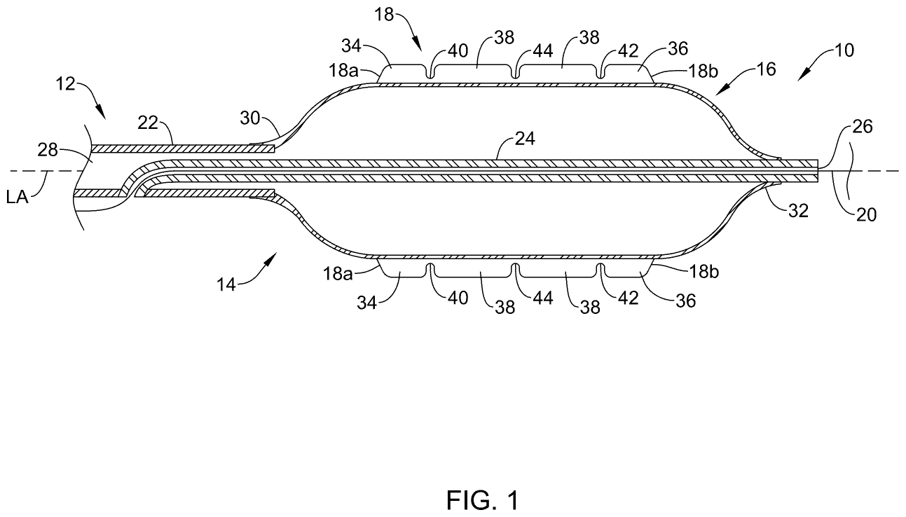

FIG. 1 is a partial cross-sectional side view of an example medical device 10. In some instances, the example medical device 10 may be utilized within a patient's vasculature in order to treat a stenosis within the vasculature. In some instances, the medical device 10 may be used to crack or cut a calcium lesion. The medical device 10 may define a longitudinal axis “LA”, as shown. The medical device 10 includes a catheter shaft 12 having a distal region 14. An inflatable balloon 16 may be secured to the distal region 14 of the catheter shaft 12. One or more monolithic blade members 18 may be mounted on or otherwise secured relative to the inflatable balloon 16. In some instances, the medical device 10 may be advanced over a guidewire 20, through the vasculature, to a target area. Once positioned at the target location in the vasculature, the inflatable balloon 16 can be inflated to exert a radially outward force on a lesion, as the monolithic blade members 18 engage the lesion. Thus, the monolithic blade members 18 may crack, cut or score the lesion to facilitate enlarging the lumen proximate the lesion. The target area may be within any suitable peripheral or cardiac vessel lumen location.

The monolithic blade members 18 may vary in number, position, and arrangement about the inflatable balloon 16. For example, the medical device 10 may include one, two, three, four, five, six, or more monolithic blade members 18 that are disposed at any position along the inflatable balloon 16 and in a regular, irregular, or any other suitable pattern. For example, in some instances, the inflatable balloon 16 may include a plurality of monolithic blade members 18 longitudinally arranged symmetrically around a circumference of the inflatable balloon 16.

The monolithic blade members 18 may be made from any suitable material such as a metal, metal alloy, polymer, metal-polymer composite, and the like, or any other suitable material. For example, the monolithic blade members 18 may be made from stainless steel, titanium, nickel-titanium alloys, tantalum, iron-cobalt-nickel alloys, or other metallic materials in some instances. The monolithic blade members 18 may have a triangular cross-sectional shape, or a square cross-sectional shape, or a rectangular cross-sectional shape, a circular cross-sectional shape or any other desired configuration.

The monolithic blade members 18 may be mounted to the inflatable balloon 16 using a variety of techniques. In some cases, each of the monolithic blade members 18 may include a base portion that includes cutouts for flexibility, where the base portion may be at least partially embedded in a polymeric member (not shown) that may itself be secured to an outer surface of the inflatable balloon 16. Additional details regarding how the monolithic blade member 18 may be secured to the outer surface of the inflatable balloon 16, including the aforementioned polymeric member, are described for example in U.S. Pat. Nos. 9,226,768; 10,046,146; 10,058,349; and 10,729,893, which are incorporated by reference in their entirety.

The inflatable balloon 16 may be made from angioplasty balloon materials including polymers such as polyethylene terephthalate (PET), polytetrafluoroethylene (PTFE), ethylene tetrafluoroethylene (ETFE), polybutylene terephthalate (PBT), polyurethane, polyvinylchloride (PVC), polyether-ester, polyester, polyamide, elastomeric polyamides, polyether block amide (PEBA), as well as other suitable materials, or mixtures, combinations, copolymers thereof, polymer/metal composites, and the like. In some instances, the inflatable balloon 16 may include a single layer of material, whereas in other instances the inflatable balloon 16 may be of a multi-layer construction, including a plurality of layers of materials. For instance, the inflatable balloon 16 may be formed as a co-extrusion or tri-layer extrusion in some instances.

The inflatable balloon 16 may be configured so that the inflatable balloon 16 includes one or more “wings” or wing-shaped regions when the inflatable balloon 16 is deflated. In some instances, the wings may be configured so that the monolithic blade members 18 can be positioned at the inward-most positions of the deflated inflatable balloon 16, with the wings of the balloon folds positioned between adjacent monolithic blade members 18. This arrangement may reduce the exposure of the monolithic blade members 18 to the blood vessel during delivery of the inflatable balloon 16 to the lesion.

The catheter shaft 12 may be a catheter shaft, similar to typical catheter shafts. For example, the catheter shaft 12 may include an outer tubular member 22 and an inner tubular member 24 extending through at least a portion of the outer tubular member 22. The outer tubular member 22 and the inner tubular member 24 may each be manufactured from a number of different materials. For example, the outer tubular member 22 and the inner tubular member 24 may be made of metals, metal alloys, polymers, metal-polymer composites or any other suitable materials.

The outer tubular member 22 and the inner tubular member 24 may be arranged in any appropriate way. For example, in some embodiments the inner tubular member 24 can be disposed coaxially within the outer tubular member 22. According to these embodiments, the inner tubular member 24 and the outer tubular member 22 may or may not be secured to one another along the general longitudinal axis of the catheter shaft 12. Alternatively, the inner tubular member 24 may follow the inner wall or otherwise be disposed adjacent the inner wall of the outer tubular member 22. In other embodiments, the outer tubular member 22 and the inner tubular member 24 may be arranged in another desired fashion.

The inner tubular member 24 may include an inner lumen 26. In at least some instances, the inner lumen 26 is a guidewire lumen for receiving the guidewire 20 therethrough. Accordingly, the medical device 10 can be advanced over the guidewire 20 to the desired location. The guidewire lumen 26 may extend along essentially the entire length of the catheter shaft 12 such that the medical device 10 resembles a traditional “over-the-wire” catheter. Alternatively, and as shown, the guidewire lumen 26 may extend along only a portion of the catheter shaft 12 such that the medical device 10 resembles a “single-operator-exchange” or “rapid-exchange” catheter.

The catheter shaft 12 may also include an inflation lumen 28 that may be used, for example, to transport inflation media to and from the inflatable balloon 16 to selectively inflate and/or deflate the inflatable balloon 16. The location and position of the inflation lumen 28 may vary, depending on the configuration of the outer tubular member 22 and the inner tubular member 24. For example, when the outer tubular member 22 surrounds the inner tubular member 24, the inflation lumen 28 may be defined within the space between the outer tubular member 22 and the inner tubular member 24. In instances in which the outer tubular member 22 is disposed alongside the inner tubular member 24, then the inflation lumen 28 may be the lumen of the outer tubular member 22.

The inflatable balloon 16 may be coupled to the catheter shaft 12 in any of a number of suitable ways. For example, the inflatable balloon 16 may be adhesively or thermally bonded to the catheter shaft 12. In some embodiments, a proximal waist 30 of the inflatable balloon 16 may be bonded to the catheter shaft 12, for example, bonded to a distal end of the outer tubular member 22, and a distal waist 32 of the inflatable balloon 16 may be bonded to the catheter shaft 12, for example, bonded to a distal end of the inner tubular member 24. The exact bonding positions, however, may vary.

Each of the monolithic blade members 18 may be considered as including a first terminal blade segment 34 at a first end of the monolithic blade member 18 and a second terminal blade segment 36 at an opposite, second end of the monolithic blade member 18. As illustrated, the first terminal blade segment 34 corresponds to a proximal-most blade segment while the second terminal blade segment 36 corresponds to a distal-most blade segment. In some instances, the first terminal blade segment 34 may correspond to a distal-most blade segment and the second terminal blade segment 36 may correspond to a proximal-most blade segment. The first terminal blade segment 34 extends from a first end 18a of the monolithic blade member 18 to a first flex point 40 that is formed in the monolithic blade member 18. The second terminal blade segment 36 extends from a second end 18b of the monolithic blade member 18 to a second flex point 42 that is formed in the monolithic blade member 18.

In some instances, the first flex point 40 and the second flex point 42, the relative position of which may be thought of as defining a first terminal blade segment length and a second terminal blade segment length, respectively, are positioned such that the first flex point 40 is within two millimeters of a first end of a lesion when the second flex point 42 is within two millimeters of a second end of the lesion. In some instances, the first terminal blade segment 34 may have a length of less than three millimeters. In some instances, the first terminal blade segment 34 may have a length of less than two millimeters. In some instances, the second terminal blade segment 36 may have a length of less than three millimeters. In some instances, the second terminal blade segment 36 may have a length of less than two millimeters.

Each of the monolithic blade members 18 may include one or more intermediate blade segments 38. As shown, each monolithic blade member 18 includes two intermediate blade segments 38, separated by a third flex point 44. In some instances, each monolithic blade member 18 may only include one intermediate blade segment 38, and thus would not include the third flex point 44. In some instances, each monolithic blade member 18 may include three or more intermediate blade segments 38, and thus may include a fourth flex point or even a fifth flex point. It will be appreciated that the number of intermediate blade segments 38 may be determined at least in part upon a desired overall length for each monolithic blade member 18. In some instances, the inflatable balloon 16 may include two or more monolithic blade members 18 each having the same overall length. In some instances, the inflatable balloon 16 may include two or more monolithic blade members 18 each having a different overall length.

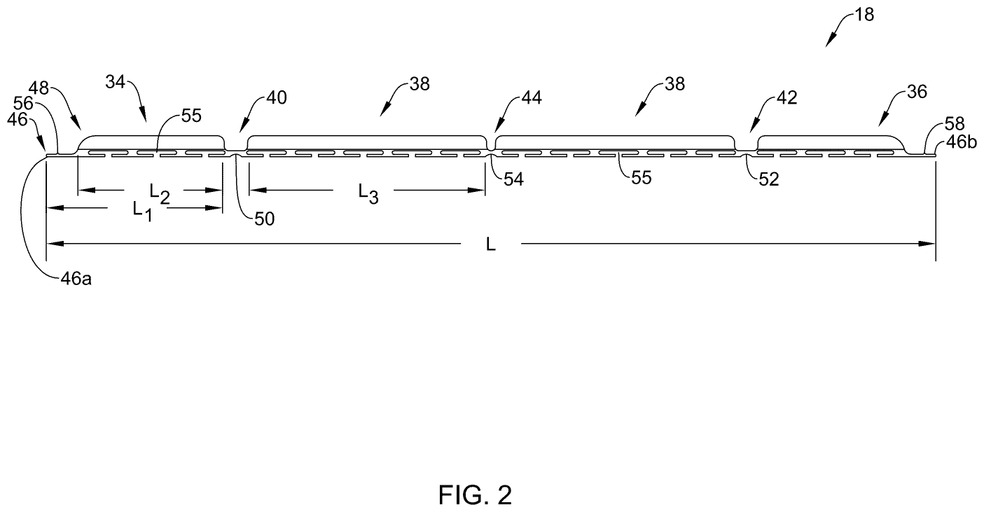

FIG. 2 is a side view of one of the monolithic blade members 18. The monolithic blade member 18 shown in FIG. 2 may be considered as being the same as the monolithic blade member 18 shown in FIG. 1. The monolithic blade member 18 includes a base portion 46 that extends from a first end 46a of the monolithic blade member 18 to a second end 46b of the monolithic blade member 18. The monolithic blade member 18 includes a blade portion 48 that extends outwardly from the base portion 46. The blade portion 48 includes the first terminal blade segment 34, the second terminal blade segment 36 and the intermediate blade segments 38, separated by the first flex point 40, the second flex point 42 and the third flex point 44, respectively.

In some instances, as shown, the first flex point 40 and the second flex point 42 may be wider than the third flex point 44 (or any additional intervening flex points). The first flex point 40 and the second flex point 42 may each be ten percent wider, or twenty percent wider, or thirty percent wider, or forty percent wider, or fifty percent wider, or sixty percent wider, or seventy percent wider, or eighty percent wider, or 90 percent wider, or 100 percent wider, or more, than the third flex point 44. In some instances, having the first flex point 40 and the second flex point 42 being wider may allow the first terminal blade segment 34 and the second terminal blade segment 36, respectively, to flex to a relatively greater angle relative to adjacent intermediate blade segments 38 without the first terminal blade segment 34 and/or the second terminal blade segment 36 hitting or otherwise being interfered with by the adjacent intermediate blade segments 38. In some instances, the first flex point 40 and/or the second flex point 42 may be adapted to permit the first terminal blade segment 34 and/or the second terminal blade segment 36 to flex to a position in which the first terminal blade segment 34 and/or the second terminal blade segment 36 forms an angle of up to 90 degrees with respect to the intermediate blade segments 38.

In some instances, the first flex point 40 may be considered as being a first gap in the blade portion 48 (e.g., a gap between adjacent portions of the cutting edge of the blade portion 48), the second flex point 42 may be considered as being a second gap in the blade portion 48 (e.g., a gap between adjacent portions of the cutting edge of the blade portion 48) and the third flex point 44 may be considered as being a third gap in the blade portion 48 (e.g., a gap between adjacent portions of the cutting edge of the blade portion 48). In some cases, as shown, the gaps extend through the blade portion 48 but do not extend through the base portion 48, meaning that the monolithic blade member 18 is a single, unitary structure, formed from a single piece of material.

In some instances, the base portion 48 may include a thinned region that corresponds to the position of the flex points 40, 42 and 44. As shown, the base portion 46 includes a first thinned region 50 that aligns with the first flex point 40, a second thinned region 52 that aligns with the second flex point 42 and a third thinned region 54 that aligns with the third flex point 44. It will be appreciated that the thinned regions 50, 52 and 54 cooperate with the gaps corresponding to the flex points 40, 42 and 44 to further improve the flexibility of the monolithic blade member 18. Flexibility of the monolithic blade member 18 helps with being able to navigate small or heavily curved portions of the vasculature as well as with allowing the terminal blade segments 34 and 36 to bend or flex further with respect to the intermediate blade segments 38.

In some instances, the base portion 48 may include a number of T-shaped slots or cutouts 55 that help to further improve the flexibility of the monolithic blade member 18. The T-shaped cutouts 55 also help to anchor the base portion 48 within a polymeric member or pad (not shown) that is itself secured to the inflatable balloon 16 in order to anchor the monolithic blade member 18 relative to the inflatable balloon 16. In some instances, the material of the polymeric member or pad flows into the T-shaped cutouts 55 to further anchor the monolithic blade member 18 relative to the inflatable balloon 16. Further details regarding the T-shaped cutouts 55 and their functionality are described in U.S. Pat. Nos. 9,226,768; 10,046,146; 10,058,349; and 10,729,893, which were previously incorporated by reference. In some cases, there will be at least one or more T-shaped cutouts 55 in each of the blade segments, including terminal blade segments.

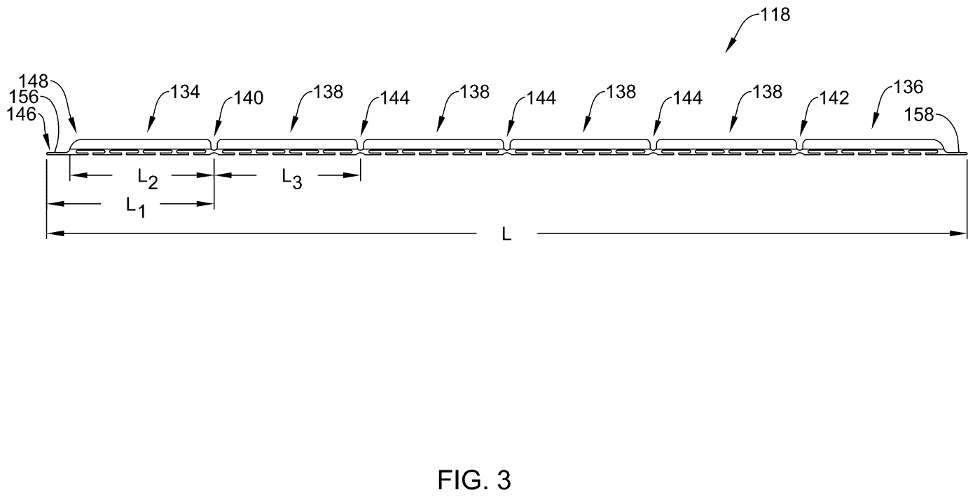

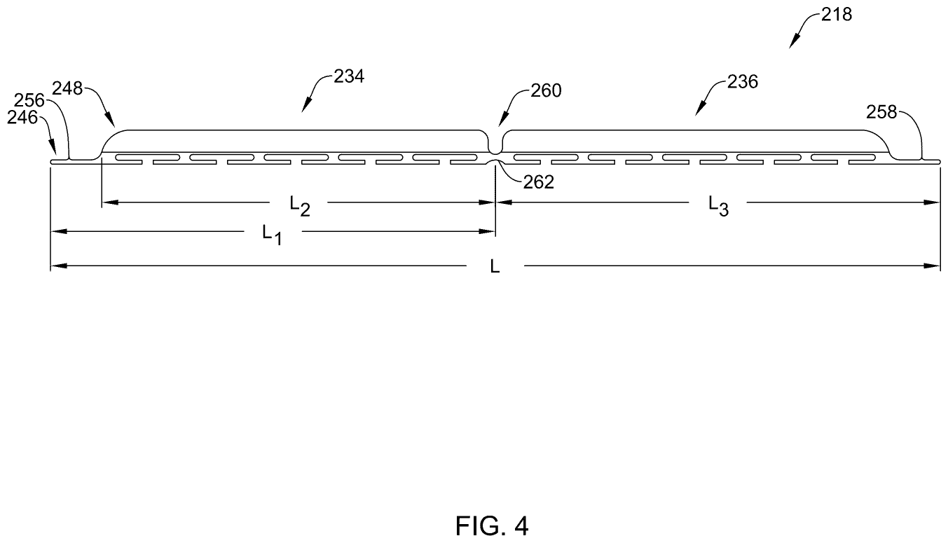

The monolithic blade member 18 has an overall length “L”. As shown, the monolithic blade member 18 has an overall length L that is equal to about 10 millimeters. FIG. 3 provides an example of a monolithic blade member having an overall length L of about 14 or 15 millimeters, and FIG. 4 provides an example of a monolithic blade member having an overall length L of about 6 millimeters. It will be appreciated that these examples are merely illustrative, as the monolithic blade members may have any desired length.

It can be seen that the base portion 46 extends beyond the blade portion 48 at the first end 46a and at the second end 46b, with the base portion 46 including a first tail piece 56 at the first end 46a and a second tail piece 58 at the second end 46b. In some instances, the additional length provided by the first tail piece 56 and the second tail piece 58 may be useful in securing the base portion 46 (and hence the monolithic blade member 18) relative to the inflatable balloon 16. In some instances, the first terminal blade segment 34 may be considered as having a length “L₁” (including the tail piece 56). In some instances, the first terminal blade segment 35 may be considered as having a length “L2” that excludes the tail piece 56. While not labeled, in some instances the second terminal blade segment 36 may be considered as having similar dimensions.

In some instances, L₁ and L₂ may each be 3 millimeters or less. In some instances, L₁ and L₂ may each be 2 millimeters or less. In a particular example, when L is 15 millimeters, L₁ may be equal to about 2 millimeters, such as 2.07 millimeters, and L₂ may be equal to about 1.75 millimeters, such as 1.74 millimeters. Each of the intermediate blade segments 38 may be considered as having a length “L₃” that is defined as the distance between the flex points located at either end of the intermediate blade segment 38, such as the first flex point 40 and the third flex point 44, or the second flex point 42 and the third flex point 44. L₃ may be adjusted to provide a desired overall length for the monolithic blade member 18. As an example, when L is equal to about 10 millimeters, each of the intermediate blade segments 38 may have a length L₃ that is equal to about 2.75 millimeters, such as 2.77 millimeters.

The monolithic blade members 18 shown in FIGS. 1 and 2 are just one example. FIGS. 3 and 4 provide additional details regarding the monolithic blade member 18. FIG. 3 shows a monolithic blade member 118 that may be used as one or more of the monolithic blade members 18 shown in FIG. 1. The monolithic blade member 118 has an overall length “L” that is equal to about 14 millimeters. The monolithic blade member 118 includes a base portion 146 and a blade portion 148 that is divided into a number of blade segments via intervening flex points. In some instances, as shown, the monolithic blade member 118 includes a first terminal blade segment 134 and a second terminal blade segment 136. The first terminal blade segment 134 may be considered as having a length “L₁”, including a first tail piece 156. The first terminal blade segment 134 may be considered as having a length “L₂” that does not include the first tail piece 156. While not labeled, in some instances the second terminal blade segment 136 may be considered as having similar dimensions.

In some instances, L₁ and L₂ may each be 3 millimeters or less. In some instances, L₁ and L₂ may each be 2 millimeters or less. In a particular example, when L is about 14 millimeters, L₁ may be equal to about 2.6 millimeters, such as 2.60 millimeters, and L₂ may be equal to about 2.25 millimeters, such as 2.23 millimeters. Each of the intermediate blade segments 38 may be considered as having a length “L₃” that is defined as the distance between the flex points located at either end of the intermediate blade segment 138, such as a first flex point 140 and a third (and additional) flex point 144. L₃ may be adjusted to provide a desired overall length for the monolithic blade member 118. In some instances, L₃ may be selected to be equal to L₂, for example.

FIG. 4 shows a monolithic blade member 218 that may be used as one or more of the monolithic blade members 18 show in FIG. 1. The monolithic blade member 218 has an overall length “L” that is equal to about 6 millimeters. The monolithic blade member 228 includes a base portion 246 and a blade portion 248 that is divided into a pair of blade segments via an intervening flex point 260. In some instances, as shown, the monolithic blade member 228 includes a first terminal blade segment 234 and a second terminal blade segment 236. The first terminal blade segment 134 may be considered as having a length “L₁”, including a first tail piece 256. The first terminal blade segment 234 may be considered as having a length “L₂” that does not include the first tail piece 256. The second terminal blade segment 236 may be considered as having a length “L₃” that is inclusive of a second tail piece 258. The base portion includes a thinned region 262 that corresponds to the flex point 260. In some instances, L₂ and L₃ may each be about 3 millimeters.

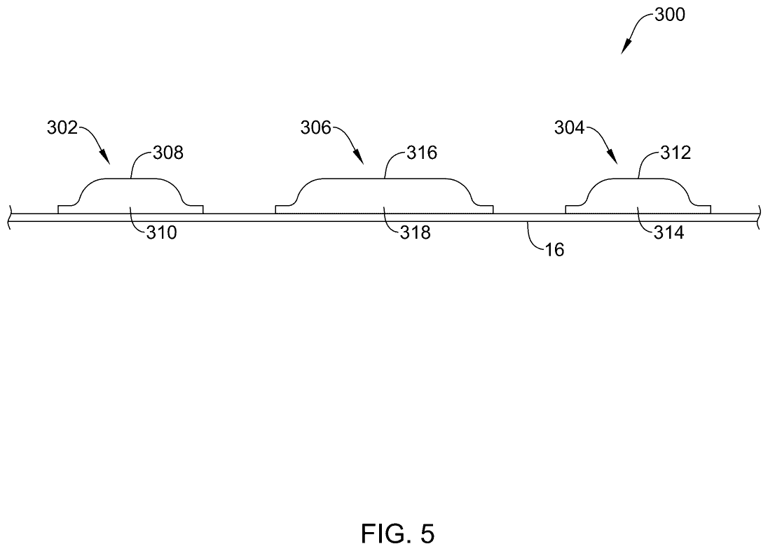

In some instances, the blade members such as the blade members 18, 118 and 218 are described as being monolithic, meaning that the blade members 18, 118 and 218 may be formed as one continuous piece of material extending an entire length of the blade members 18, 118, 218. In other words, the terminal blade segments and the intermediate blade segment(s) may be formed of a singled continuous piece of material. In some instances, some blade assemblies may include a plurality of separate blade segments, with each of the plurality of separate blade segments axially aligned with one another. Some of the plurality of separate blade segments may be disposed at an acute angle relative to adjacent blade segments. In some instances, the blade segments may be secured within a polymeric pad that is itself adapted to be secured to an outer surface of the inflatable balloon 16. Each blade segment may be secured within its own polymeric pad, or at least some of the blade segments may be secured within the same polymeric pad. FIGS. 5 through 7 provide examples of illustrative blade assembles.

FIG. 5 shows a blade assembly 300 mounted on the inflatable balloon 16. The blade assembly 300 includes a first terminal blade segment 302, a second terminal blade segment 304 and an intervening blade segment 306. It will be appreciated that the first terminal blade segment 302, the second terminal blade segment 304 any intervening blade segments 306 (only one is shown) are formed as distinct elements that are not directly attached to each other. In some instances, the first terminal blade segment 302, the second terminal blade segment 304 and the intervening blade segment 306 may be formed as a unitary structure, such as that shown with respect to FIGS. 2, 3 and 4, and the individual blade segments may be snapped apart, or otherwise separated from one another, before being embedded within a polymeric pad for securement to the inflatable balloon 16.

In some instances, the first terminal blade segment 302 and the second terminal blade segment 304 may each have a length of less than 3 millimeters. In some instances, each of the first terminal blade segment 302 and the second terminal blade segment 304 may each have a length of less than 2 millimeters. In some instances, the intervening blade segment 306 may have a length that is greater than 3 millimeters, while the first and second terminal blade segments 302, 304 may have a length that is less than 3 millimeters.

In some instances, each of the first terminal blade segment 302, the second terminal blade segment 304 and the intervening blade segment 306 may each be secured to a single piece polymeric pad for securing the blade assembly 300 to an inflatable balloon such as the inflatable balloon 16. In some instances, each of the first terminal blade segment 302, the second terminal blade segment 304 and the intervening blade segment 306 may be individually secured to its own distinct polymeric pad segment spaced apart from one another.

The first terminal blade segment 302 includes a blade portion 308 and a base portion 310. The second terminal blade segment 304 includes a blade portion 312 and a base portion 314. The intervening blade segment 306 includes a blade portion 316 and a base portion 318. It will be appreciated that the blade portions 308, 312 and 316 may be adapted to cut or crack a lesion when a medical device including the blade assembly 300 is advanced through the vasculature to be positioned proximate a lesion, and the inflatable balloon 16 is inflated to drive the blade assembly 300 in a radially outward direction in order to engage the lesion. The base portion 310, the base portion 314 and the base portion 318 may each be adapted to be embedded within a polymeric pad for securement to the inflatable balloon 16.

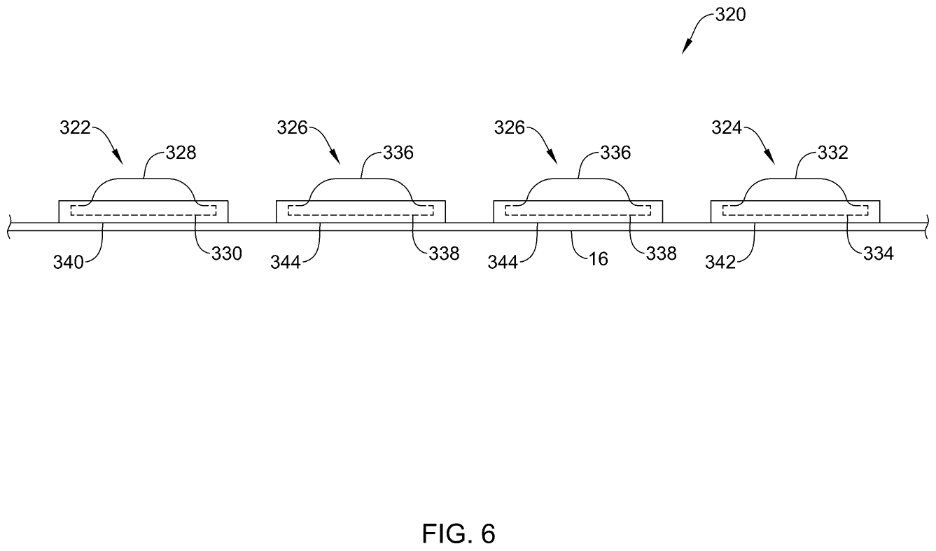

FIG. 6 shows a blade assembly 320 mounted on the inflatable balloon 16. The blade assembly 320 includes a first terminal blade segment 322, a second terminal blade segment 324 and several intervening (two are shown) blade segments 326. While a pair of intervening blade segments 326 are shown, in some instances the blade assembly 320 may only include one intervening blade segment 326. In some instances, the blade assembly 320 may include three, four or more intervening blade segments 326, depending on the desired overall length of the blade assembly 320.

In some instances, the first terminal blade segment 322, the second terminal blade segment 324 and the intervening blade segments 326 may be formed as a unitary structure, such as that shown with respect to FIGS. 2, 3 and 4, and the individual blade segments may be snapped apart, or otherwise separated from one another, before being embedded within a polymeric pad for securement to the inflatable balloon 16. For example, the blade segments 322, 324, 326 may be made from stainless steel, titanium, nickel-titanium alloys, tantalum, iron-cobalt-nickel alloys, or other metallic materials in some instances. The blade segments 322, 324, 326 may have a cross sectional shape of a triangle, square, rectangle, circle or any desired configuration. In some instances, the first terminal blade segment 322, the second terminal blade segment 324 and the intervening blade segments 326 may each be equal in length. For example, each of the first terminal blade segment 322, the second terminal blade segment 324 and the intervening blade segments 326 may have a length of less than 3 millimeters. As another example, each of the first terminal blade segment 322, the second terminal blade segment 324 and the intervening blade segments 326 may have a length of less than 2 millimeters.

The first terminal blade segment 322 includes a blade portion 328 and a base portion 330. The second terminal blade segment 324 includes a blade portion 332 and a base portion 334. Each intervening blade segment 326 includes a blade portion 336 and a base portion 338. The base portion 330 is shown embedded within a polymeric pad segment 340, the base portion 334 is shown embedded within a polymeric pad segment 342, and each base portion 338 is shown embedded within a polymeric pad segment 344. In some instances, having each distinct blade segment embedded in its own distinct polymeric pad segment may be beneficial in providing additional flexibility.

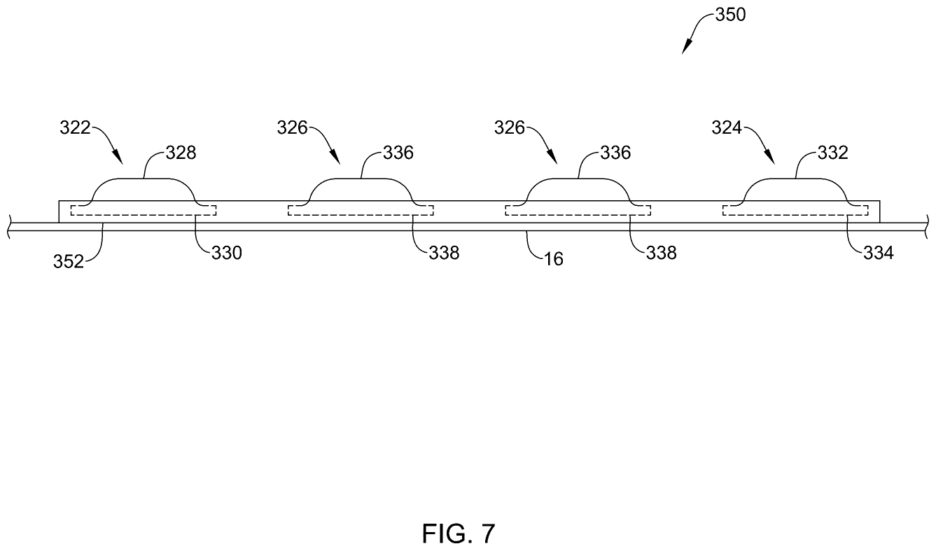

FIG. 7 shows a blade assembly 350 mounted on the inflatable balloon 16. The blade assembly 350 includes the first terminal blade segment 322, the second terminal blade segment 324 and several intervening (two are shown) blade segments 326. While a pair of intervening blade segments 326 are shown, in some instances the blade assembly 350 may only include one intervening blade segment 326. In some instances, the blade assembly 350 may include three, four or more intervening blade segments 326, depending on the desired overall length of the blade assembly 350.

The first terminal blade segment 322 includes the blade portion 328 and the base portion 330. The second terminal blade segment 324 includes the blade portion 332 and the base portion 334. Each intervening blade segment 326 includes the blade portion 336 and the base portion 338. The base portions 330, 334 and 338 are each embedded within a unitary or single polymeric pad 352. The polymeric pad 352 may be secured to the outer surface of the inflatable balloon 16 in order to form a medical device.

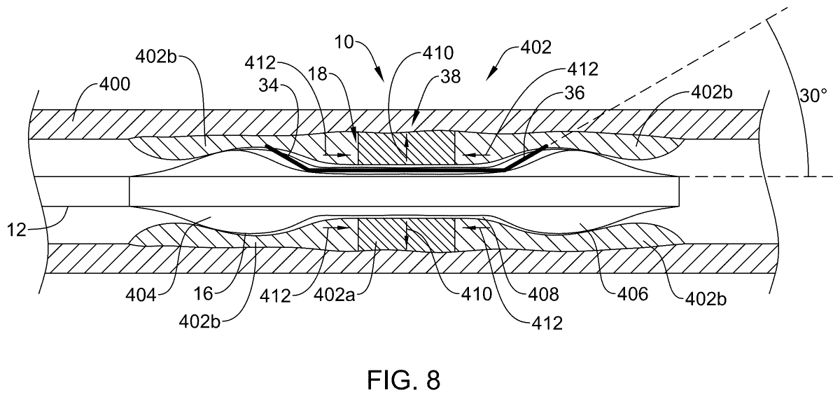

FIG. 8 shows the medical device 10 deployed within a blood vessel 400 proximate a lesion 402. In some instances, the lesion 402 may not be uniform in size or composition. In some instances, the lesion 402 may include a middle section 402a that is more heavily calcified and thus less compliant. The lesion 402 may include end sections 402b on either side of the middle section 402a. The end sections 402b may be less calcified and thus may be more compliant. In some instances, when the inflatable balloon 16 is inflated, the intermediate blade segments 38, driven by an intermediate portion 408 of the inflatable balloon 16, may exert more of a radial force on the middle section 402a, as indicated by arrows 408, and the terminal blade segments 34 and 36 may exert more of an axial force on the middle section 402, as indicated by arrows 412.

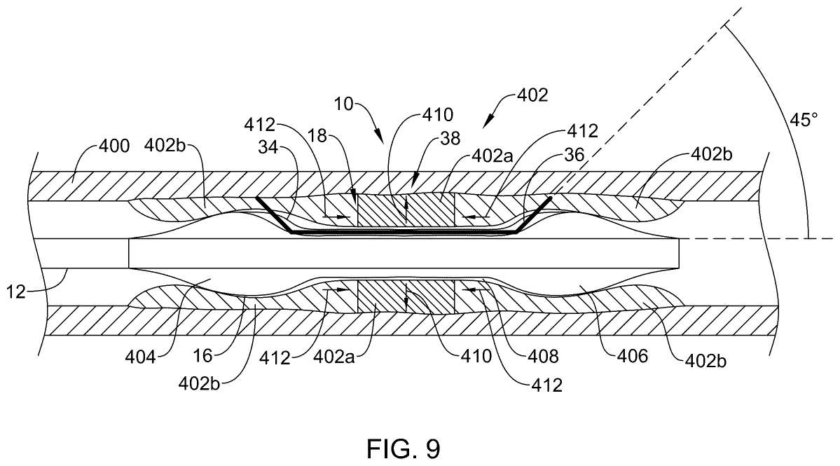

It will be appreciated that the geometry of the lesion 402, particularly the middle section 402a, may cause the inflatable balloon 16 to achieve a “dog bone” shape as the blade assembly 18 engages the lesion 402, with a first region 404 of the inflatable balloon 16 and a second region 406 of the inflatable balloon 16 able to achieve a greater diameter than an intermediate region of the inflatable balloon 16. This means that the first terminal blade segment 34 and the second terminal blade segment 36 may be able to achieve an angle, with respect to the intermediate blade segments 38, of 30 degrees or more. In some cases, as shown in FIG. 9, the first terminal blade segment 34 and the second terminal blade segment 36 may be able to achieve an angle, with respect to the intermediate blade segments 38, of 45 degrees or more. These angles may be achievable, for example, by having terminal blade segments 34 and 36 that extend beyond the periphery of the non-compliant or restrictive section of the lesion 402 (such as the middle section 402a of the lesion 402).

A method of treating the lesion 402 may include advancing a medical device such as the medical device 10 through the blood vessel 400 to a position proximate the lesion 402. In some instances, the dimensions of the lesion 402 may be estimated or determined using fluoroscopy and a contrast agent. In some instances, the medical device 10 may be selected such that the monolithic blades 18 (or the blade assemblies 300, 320 and 350) forming part of the medical device 10 are several millimeters longer than an axial length of the non-compliant or restrictive section of the lesion 402 (such as the middle section 402a). In some instances, the lesion 402 may be longer than the monolithic blades 18 or the blade assemblies 300, 320, 350. As noted, the medical device includes the inflatable balloon 16 and the monolithic blade 18. The monolithic blade 18 includes the base portion 46 and the blade portion 48. The blade portion 48 is divided into a first blade segment, a second blade segment and an intervening third blade segment. The medical device 10 is positioned such that the first blade segment extends in a first direction beyond a periphery of the resistive section 402a of the lesion 402 (e.g., beyond a first terminal extent of the resistive section 402a of the lesion 402) and the second blade segment extends in a second direction beyond the periphery of the restrictive section 402a of the lesion 402 (e.g., beyond a second terminal extent of the restrictive section 402a of the lesion 402). The inflatable balloon 16 is inflated to exert the monolithic blade 18 towards the lesion 402. At least one of the first blade segment and the second blade segment flexes to an angle of 30 degrees or greater than relative to the intervening third blade segment. In some instances, at least one of the first blade segment and the second blade segment flexes to an angle of 45 degrees or greater than with respect to the third blade segment when the inflatable balloon 16 is inflated.

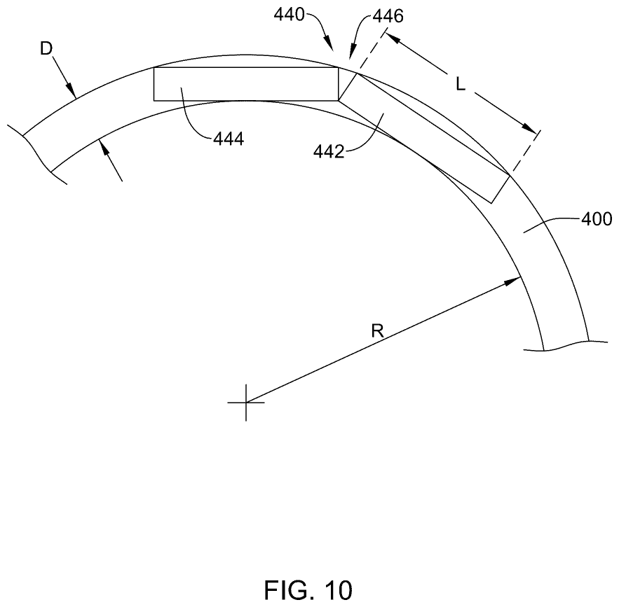

In addition to being able to provide additional cracking forces at a periphery of the resistive section 402a of the lesion 402, having shorter blade segments in general may improve deliverability of the medical device 10. FIG. 10 schematically shows a curved portion of the blood vessel 400, having a radius of curvature “R” measured to a mid-point of the blood vessel 400. A monolithic blade 440 (the corresponding inflatable balloon is not shown) including a first blade segment 342 and a second blade segment 444, separated by a flex point 446, is shown disposed within the curved portion of the blood vessel 400. Each of the first blade segment 442 and the second blade segment 444 have a length “L”. As will be appreciated, as the radius of curvature R increases, the length L that will still permit the monolithic blade 440 to pass through the curved vessel will increase. As the radius of curvature R decreases, the length L that will still permit the monolithic blade 440 to pass through the curved vessel decrease. Of course, the diameter of the blood vessel 400, as indicated by a diameter “D”, will also play a part in what length of monolithic blade 440 will pass through a vessel having a particular radius of curvature R.

Having thus described several illustrative embodiments of the present disclosure, those of skill in the art will readily appreciate that yet other embodiments may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A medical device, comprising:
a catheter shaft having a distal region;
an inflatable balloon secured to the distal region; and
a monolithic blade member extending longitudinally relative to the inflatable balloon, the monolithic blade member secured relative to an outer surface of the inflatable balloon, the monolithic blade member comprising:
a base portion extending from a first end of the monolithic blade member to a second end of the monolithic blade member;
a blade portion extending outwardly from the base portion, the blade portion including:
a first terminal blade segment extending from the first end of the monolithic blade member to a first flex point formed in the monolithic blade member, the first terminal blade segment having a first terminal blade segment length;
a second terminal blade segment extending from the second end of the monolithic blade member to a second flex point formed in the monolithic blade member, the second terminal blade member having a second terminal blade segment length; and
at least one intermediate blade segment extending between the first flex point and the second flex point, each of the at least one intermediate blade segments having an intermediate blade segment length that is greater than the first terminal blade member segment length and the second terminal blade member segment length.

2. The medical device of claim 1, wherein the terminal blade segment length allows the first flex point to be within two millimeters of a first end of a lesion when the second flex point is within two millimeters of an opposing second end of the lesion.

3. The medical device of claim 1, wherein the first flex point comprises a first gap in the blade portion.

4. The medical device of claim 1, wherein the second flex point comprises a second gap in the blade portion, with the base portion extending without any gap.

5. The medical device of claim 1, wherein the first terminal blade segment length is equal to the second blade segment length with the base portion extending without any gap.

6. The medical device of claim 1, wherein the first terminal blade segment has a length of less than 3 millimeters.

7. The medical device of claim 1, wherein the first terminal blade segment has a length of less than 2 millimeters.

8. The medical device of claim 7, wherein the second terminal blade segment has a length of less than 2 millimeters.

9. The medical device of claim 6, wherein the second terminal blade segment has a length of less than 3 millimeters.

10. The medical device of claim 1, further comprising one or more additional monolithic blade members extending longitudinally along the outer surface of the inflatable balloon, each of the one or more additional monolithic blade members comprising:
a base portion extending from a first end of the monolithic blade member to a second end of the monolithic blade member;
a blade portion extending outwardly from the base portion, the blade portion including:
a first terminal blade segment extending from the first end of the monolithic blade member to a first flex point formed in the monolithic blade member, the first terminal blade segment having a first terminal blade segment length;
a second terminal blade segment extending from the second end of the monolithic blade to and a second flex point formed in the monolithic blade member, the second terminal blade segment having a second terminal blade segment length; and
at least one intermediate blade segment extending between the first flex point and the second flex point, the at least one intermediate blade segment having an intermediate blade segment length that is greater than the first terminal blade segment length and the second terminal blade segment length.

11. A medical device, comprising:
an inflatable balloon secured to a distal region of a catheter shaft having a longitudinal axis;
a plurality of monolithic blade members secured to the inflatable balloon and extending longitudinally with respect to the inflatable balloon, each of the plurality of monolithic blade members comprising:
a base portion extending from a first end of the monolithic blade member to a second end of the monolithic blade member;
a blade portion extending outwardly from the base portion, the blade portion including:
a first terminal blade segment extending from the first end of the monolithic blade member to a first flex point formed in the monolithic blade member, the first terminal blade segment having a length of less than 3 millimeters;
a second terminal blade segment extending from the second end of the monolithic blade member to a second flex point formed in the monolithic blade member, the second terminal blade segment having a length of less than 3 millimeters; and
at least one intermediate blade segment extending between the first flex point and the second flex point, each of the at least one intermediate blade segments having a length of greater than 3 millimeters.

12. The medical device of claim 11, wherein the plurality of monolithic blade members comprise four blade members, each arranged circumferentially spaced about 90 degrees from a closest neighbor monolithic blade member.

13. The medical device of claim 11, wherein for each of the plurality of monolithic blade members, the first pivot point comprises a first gap in the blade portion.

14. The medical device of claim 11, wherein for each of the plurality of monolithic blade members, the second pivot point comprises a second gap in the blade portion, with the base portion extending without any gap.

15. The medical device of claim 11, wherein:
the first flex point is adapted to enable the first terminal blade segment to be flexed at an angle greater than 30 degrees from the longitudinal axis; and
the second flex point is adapted to enable the second terminal blade segment to be flexed at an angle greater than 30 degrees from the longitudinal axis.

16. The medical device of claim 15, wherein the first flex point is adapted to enable the first terminal blade segment to be flexed at an angle greater than 45 degrees from the longitudinal axis.

17. The medical device of claim 16, wherein the second flex point is adapted to enable the second terminal blade segment to be flexed at an angle greater than 45 degrees from the longitudinal axis.

18. The medical device of claim 15, wherein the at least one intermediate blade segment extends parallel to the longitudinal axis while the first terminal blade segment and the second terminal blade segment are flexed at an angle greater than 30 degrees from the longitudinal axis.

19. A method for treating a lesion within a blood vessel, the method comprising:
advancing a medical device through the blood vessel to a position proximate the lesion, the medical device including
an inflatable balloon;
a monolithic blade including a base portion extending from a first end of the monolithic blade member to a second end of the monolithic blade member and a blade portion extending outwardly from the base portion, the blade portion including:
a first blade segment extending from the first end of the monolithic blade member to a first flex point formed in the monolithic blade member;
a second blade segment extending from the second end of the monolithic blade member to a second flex point formed in the monolithic blade member; and
a third blade segment extending between the first blade segment and the second blade segment;
positioning the medical device such that the first blade segment extends distally of the lesion and the second blade segment extends proximally of the lesion; and
inflating the inflatable balloon in order to exert the monolithic blade towards the lesion, at least one of the first blade segment and the second blade segment flexing to an angle greater than 30 degrees relative to the third blade segment.

20. The method of claim 19, wherein at least one of the first blade segment and the second blade segment flexes to an angle greater than 45 degrees with respect to the third blade segment when the inflatable balloon is inflated.
